(19) United States
(12) Patent Application Publication
(10) Pub. No.: US 2025/0204951 A1
(43) Pub. Date: Jun. 26, 2025

(54) METHODS AND DEVICES OF ACCESS SHEATH SYSTEMS

(71) Applicant: Silk Road Medical, Inc., Sunnyvale, CA (US)

(72) Inventor: Zack Tegels, Sunnyvale, CA (US)

(21) Appl. No.: 18/849,433

(22) PCT Filed: Mar. 21, 2023
(86) PCT No.: PCT/US2023/064772

§ 371 Date: Sep. 20, 2024

Related U.S. Application Data
(60) Provisional application No. 63/322,065, filed on Mar. 21, 2022.

(51) Int. Cl. A61B 17/34; A61M 25/06

(52) U.S. Cl. CPC A61B 17/3423; A61B 17/3496; A61B 2017/3456; A61B 2017/3486; A61M 2025/0687

(57) ABSTRACT

An access sheath system is described that can include an access sheath and a dilator. For example, the access sheath can be sized and shaped to be introduced into an artery and include a protective distal end having an atraumatic surface. The dilator can include an atraumatic tip and a guide sheath. For example, the atraumatic tip can have a proximal portion that is formed to extend along a sheath passageway of the guide sheath and a distal portion having a tapered distal surface. The atraumatic tip can be formed to mate with and cover at least a part of a distal end of the guide sheath to protect the artery from the distal end of the guide sheath.

### CROSS-REFERENCE TO RELATED APPLICATION

The current application claims priority under 35 U.S.C. § 119 (e) to U.S. Provisional patent application Ser. No. 63/322,065, filed on Mar. 21, 2022, and entitled “METHODS AND DEVICES OF ACCESS SHEATH SYSTEMS,” which is incorporated by reference herein in its entirety.

### BACKGROUND

Interventional procedures are performed to treat vascular disease, for example stenosis, occlusions, aneurysms, or fistulae. Interventional procedures are also used to perform procedures on organs or tissue targets that are accessible via blood vessels, for example denervation or ablation of tissue to intervene in nerve conduction, embolization of vessels to restrict blood flow to tumors or other tissue, and delivery of drugs, contrast, or other agents to intra or extravascular targets for therapeutic or diagnostic purposes. Interventional procedures are typically divided into coronary, neurovascular, and peripheral vascular categories. Most procedures are performed in the arterial system via an arterial access site.

Methods for gaining arterial access to perform these procedures are well-established, and fall into two broad categories: percutaneous access and surgical cut-down. The majority of interventional procedures utilize a percutaneous access. For this access method, a needle puncture is made from the skin, through the subcutaneous tissue and muscle layers to the vessel wall, and into the vessel itself. Vascular ultrasound is often used to image the vessel and surrounding structures, and facilitate accurate insertion of the needle into the vessel. In some instances, a micro-puncture or micro access technique is used whereby the vessel is initially accessed by a small gauge needle, and successively dilated up by a 4F micropuncture cannula through which the sheath guidewire is placed. Once the guidewire is placed, an access sheath and sheath dilator can be inserted over the guidewire into the artery.

In a surgical cut-down, a skin incision is made and tissue is dissected away to the level of the target artery. Depending on the size of the artery and of the access device, an incision is made into the wall of the vessel with a blade, or the vessel wall is punctured directly by an access needle, through which a sheath guide wire is placed. As above, the access sheath and sheath dilator are inserted into the artery over the sheath guide wire. Once the access sheath is placed, the dilator and sheath guide wire are removed. Devices can now be introduced via the access sheath into the artery and advanced using standard interventional techniques and fluoroscopy to the target site to perform the procedure.

Access to the target site is accomplished from an arterial access site that is entered from the skin. For example, some endovascular devices are specifically designed for a femoral access site. Additionally, other access sites include the radial, brachial, carotid, and axillary arteries. These access sites involve smaller arteries compared to the femoral artery and may include tortuous segments and some distance between the access and target sites.

A concern and potential issue when inserting an access device into a vessel include causing unwanted damage to the vessel. For example, during puncture enlargement and access into a vessel, an access sheath can be used that can be rigid and include an angled and/or sharp distal end that can cause vessel dissection and/or damage. Such vessel dissection and damage can result in procedure complications, which can harm the patient and prolong the procedure.

### SUMMARY

Aspects of the current subject matter can include embodiments of an access sheath system including various embodiments of a dilator. In one aspect, the access sheath system includes an access sheath including an elongated sheath body that is sized and shaped to be introduced into an artery. The access sheath can include a protective distal end having an atraumatic surface, and the elongated sheath body can include an inner lumen extending between a proximal sheath end and a distal sheath end. The access sheath system can further include a dilator including an atraumatic tip and a guide sheath. The atraumatic tip can include a proximal portion and a distal portion. The proximal portion of the atraumatic tip can be formed to extend along a sheath passageway of the guide sheath. The distal portion of the atraumatic tip can include a tapered distal surface, and the atraumatic tip can be formed to mate with and cover at least a part of a distal end of the guide sheath to protect the artery from the distal end of the guide sheath.

In some variations one or more of the following features can optionally be included in any feasible combination. The access sheath system can further include a guidewire sized to extend along at least a dilator passageway of the atraumatic tip. The atraumatic tip of the dilator can include an inflatable balloon tip element. The inflatable balloon tip element can include a stepped proximal surface that is formed to mate against and cover the distal end of the guide sheath when the inflatable balloon is in an inflated state. The inflatable balloon tip element can include a proximal section having a first outer diameter and a distal section having a second outer diameter, the first outer diameter can be smaller than the second outer diameter, and the proximal section can be formed to couple to the sheath passageway.

The atraumatic tip can be moveable relative to the guide sheath and along a longitudinal axis of the guide sheath for forming a first position and a second position of the dilator. The first position can include a proximal surface of the atraumatic tip being mated against and at least partially covering the distal end of the guide sheath. The proximal surface can be part of a stepped proximal surface of the atraumatic tip, and the stepped proximal surface can be proximal to the tapered surface. The stepped proximal surface can be positioned a distance away from the distal end of the guide sheath when the dilator is in the second position. The second position can include a flexible extension of the atraumatic tip being radially expanded and positioned along an outer surface of the distal end of the guide sheath. The sheath body of the access sheath can include at least one arm that, when pulled in a direction away from a longitudinal axis of the sheath body, separates the sheath body into more than one part.

In another aspect, a dilator for use with an access sheath system can include a guide sheath having a sheath passageway. The dilator can further include an atraumatic tip having a proximal portion and a distal portion. The proximal portion can be formed to extend along the sheath passageway, and the distal portion of the atraumatic tip can include a tapered distal surface. The atraumatic tip can be formed to mate with and cover at least a part of a distal end of the guide sheath to protect an artery from the distal end of the guide sheath.

In some variations one or more of the following features can optionally be included in any feasible combination. The atraumatic tip can include a dilator passageway that allows a guidewire to extend therealong and through the atraumatic tip. The atraumatic tip of the dilator can include an inflatable balloon tip element. The inflatable balloon tip element can include a stepped proximal surface that is formed to mate against and cover the distal end of the guide sheath when the inflatable balloon is an inflated state. The inflatable balloon tip element can include a proximal section having a first outer diameter and a distal section having a second outer diameter, the first outer diameter can be smaller than the second outer diameter, and the proximal section can be formed to couple to the sheath passageway. The atraumatic tip can be moveable relative to the guide sheath and along a longitudinal axis of the guide sheath for forming a first position and a second position. The first position can include a proximal surface of the atraumatic tip being mated against and at least partially covering the distal end of the guide sheath. The proximal surface can be part of a stepped proximal surface of the atraumatic tip, and the stepped proximal surface can be proximal to the tapered surface. The stepped proximal surface can be positioned a distance away from the distal end of the guide sheath when the dilator is in the second position. The second position can include a flexible extension of the atraumatic tip being radially expanded and positioned along an outer surface of the distal end of the guide sheath.

In another interrelated aspect of the current subject matter, a method includes advancing an access sheath into an artery. The access sheath can include a protective distal end having an atraumatic surface, and the elongated sheath body can include an inner lumen extending between a proximal sheath end and a distal sheath end. The method can further include advancing a dilator along the inner lumen of the access sheath. The dilator can include an atraumatic tip and a guide sheath. The atraumatic tip can have a proximal portion and a distal portion. The proximal portion can be formed to extend along a sheath passageway of the guide sheath. The distal portion of the atraumatic tip can include a tapered distal surface, and the atraumatic tip can be formed to mate with and at least partially cover a distal end of the guide sheath to protect the artery from the distal end of the guide sheath.

In some variations one or more of the following features can optionally be included in any feasible combination. The method can further include advancing a guidewire along a dilator passageway of the atraumatic tip. The atraumatic tip of the dilator can include an inflatable balloon tip element. The inflatable balloon tip element includes a stepped proximal surface that is formed to mate against and cover the distal end of the guide sheath when the inflatable balloon is an inflated state. The inflatable balloon tip element includes a proximal section having a first outer diameter and a distal section having a second outer diameter, the first outer diameter being smaller than the second outer diameter, and the proximal section coupling to the sheath passageway of the guide sheath. The method can further include moving the atraumatic tip relative to the guide sheath and along a longitudinal axis of the guide sheath for forming a first position or a second position. The first position can include a proximal surface of the atraumatic tip being mated against and at least partially covering the distal end of the guide sheath. The proximal surface can be part of a stepped proximal surface of the atraumatic tip, and the stepped proximal surface can be proximal to the tapered surface. The stepped proximal surface can be positioned a distance away from the distal end of the guide sheath when the dilator is in the second position. The second position can include a flexible extension of the atraumatic tip being radially expanded and positioned along an outer surface of the distal end of the guide sheath. The sheath body of the access sheath can include at least one arm that, when pulled in a direction away from a longitudinal axis of the sheath body, separates the sheath body into more than one part.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1A illustrates a side view of an embodiment of an access sheath system including an access sheath, a dilator, and a guidewire.

FIG. 1B illustrates the access sheath of FIG. 1A being used to access a vessel for performing a procedure.

FIG. 2A illustrates a side cross-section view of another embodiment of the access sheath system including another embodiment of the access sheath and dilator.

FIG. 2B illustrates a side view of the access sheath system of FIG. 2A including an embodiment of a guide sheath.

FIG. 3A illustrates a partial side cross-section view of an embodiment of the dilator including a balloon tip element and a guide sheath.

FIG. 3B illustrates a partial side view of the balloon tip element of FIG. 3A.

FIG. 4A illustrates a partial side view of an embodiment of a dilator in a first position, the dilator including a tapered tip element and an embodiment of the guide sheath.

FIG. 4B illustrates a partial side view of the dilator of FIG. 4A in a second position.

FIG. 4C illustrates a partial side section view of the dilator in the second position of FIG. 4B.

FIG. 5A illustrates a side section view of another embodiment of the dilator in a first position, the dilator including a flexible tapered tip element and an embodiment of the guide sheath.

FIG. 5B illustrates a partial side section view of the dilator of FIG. 5A in a second position.

When practical, similar reference numbers denote similar structures, features, or elements.

### DETAILED DESCRIPTION

Disclosed are various embodiments of an access sheath system that enable safe and effective access to vasculature, such as to perform a variety of treatments accessed via the vasculature of the patient. For example, the access sheath systems disclosed herein can include one or more of an access sheath, a dilator and a guidewire. Additionally, embodiments of the access sheath and the dilator are described herein that include features for ensuring safe access into and along vasculature. For example, some embodiments of the access sheath and the dilator described herein include features to prevent unwanted damage to a vessel, such as to a vessel wall during and/or after insertion of the access sheath into the vessel.

The various access sheath systems disclosed herein, including the various access sheaths, dilators and guidewires, can be used with any of a variety of vasculature of a patient, including the femoral radial, brachial, carotid, and axillary arteries. As such, any examples of the devices, systems, and/or methods disclosed herein that are described in relation to a specific access point and/or a specific vasculature are not limited to such specific access point and/or specific vasculature.

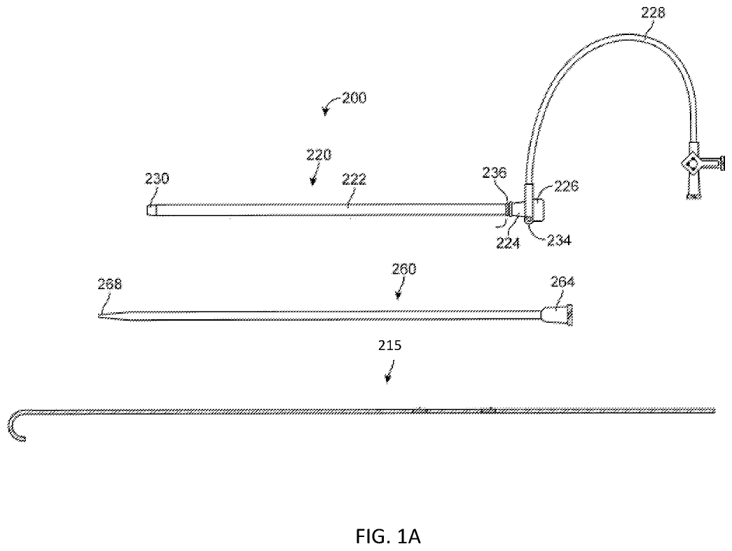

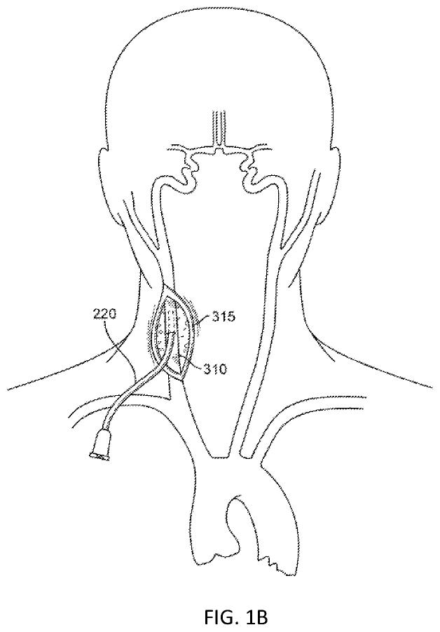

FIG. 1A shows a first embodiment of an access sheath system 200 including an access sheath for inserting into a vessel over a guidewire. When inserted into the vessel, the access sheath enables or allows introduction of at least one interventional device into the vessel via an inner lumen of the access sheath for the purpose of performing an interventional procedure on a region of the vasculature. As shown in FIG. 1A, the access sheath system 200 includes an access sheath 220, a dilator 260, and a guidewire 215. The access sheath 220, dilator 260 and guidewire 215 are all adapted to be introduced via an access site, such as a carotid puncture into the carotid artery. The access site may be accomplished percutaneously or via a surgical cut down.

In an embodiment, some or all of the components of the access sheath system 200 may be combined into one access system kit such as by combining one or more of the access sheath 220, dilator 260 and guidewire 215 into a single, package, container or a collection of containers that are bundled together.

FIG. 1B shows an example procedure using the access sheath system 200, such as the access sheath 220 being used to access a common carotid artery 310 for a carotid stenting procedure. As shown in FIG. 1B, the access sheath 220 can be inserted into the common carotid artery 310 via a surgical cut down 315. The access sheath 220 can include an inner lumen with openings at proximal and distal ends or regions of the access sheath 220. With a distal portion of the access sheath 220 in the carotid artery and a proximal portion external to the patient, the inner lumen can provide a passageway to insert one or more interventional devices into the artery for performing various procedures.

With reference again to FIG. 1A, an embodiment of an arterial access sheath 220 includes an elongated sheath body 222 and a proximal adaptor 224 at a proximal end of the sheath body 222. The sheath body 222 is the portion of the arterial access sheath 220 that is sized and shaped to be inserted into the artery and wherein at least a portion of the sheath body 222 is actually inserted into the artery during a procedure. The proximal adaptor 224 can include a hemostasis valve 226 and an elongated flush line 228 having an internal lumen that communicates with an internal lumen of the sheath body 222. The proximal adaptor 224 may have a larger diameter or cross-sectional dimension than the sheath body 222. The hemostasis valve 226 can communicate with an internal lumen of the sheath body 222 to allow introduction of devices therein while preventing or minimizing blood loss via the inner lumen during the procedure. For example, the hemostasis valve 226 can include a static seal-type passive valve, an adjustable-opening valve (e.g., a Tuohy-Borst valve), or a rotating hemostasis valve. In an embodiment, the sheath body 222 has an outer diameter that is approximately 5 to 9 French, or 6 or 7 French. In an embodiment, the sheath body 222 has an inner lumen diameter of approximately 0.087″ and an outer diameter of approximately.104″ corresponding to a 6 French sheath size. In another embodiment, the sheath body 222 has an inner lumen diameter of approximately 0.113″ and an outer diameter of approximately 0.136″ corresponding to an 8 French sheath size. Other sizes and dimensions of the sheath body 222 are within the scope of this disclosure.

The access sheath 220 may also include a radiopaque marker 230, such as a radiopaque marker 230 positioned adjacent a distal end of the sheath body 222, as shown in FIG. 1A. The radiopaque marker 230 can include a metal band, for example a platinum iridium alloy embedded near the distal end of the sheath body 222. Alternately, material forming a part of the sheath body 222 (e.g., a distal end of the sheath body 222) can include radiopaque material, such as a barium polymer or tungsten polymer blend.

A shown in FIG. 1A, the dilator 260 can include an elongated body that can be inserted into the vessel and enable smooth insertion of the access sheath 220 through a puncture site in the vessel wall. Thus, the distal end of the dilator 260 is generally tapered to allow the dilator 260 to be inserted over the guidewire 215 into the artery, and to dilate the access site to a larger diameter for insertion of the access sheath 220. To accommodate these functions, the dilator 260 can include a tapered end 268 with a radiused leading edge. For example, the dilator 260 can be secured to the access sheath 220 when assembled for insertion into the artery. The dilator 260 can include a proximal hub or cap 264, such as for coupling to a corresponding structure of the arterial access sheath 220 (e.g., the hemostasis valve 226). An inner passageway of the dilator 260 can accommodate a guidewire 215. For example, the dilator 260 can include a diameter of approximately 0.037 inch (in) to approximately 0.041 in. In some embodiments, the dilator 260 can include one or more radiopaque markers 230, such as at a distal end. In one variation, the radiopaque marker 230 is a section of tungsten loaded Pebax or polyurethane that is heat welded to the distal end of the dilator 260. Other radiopaque materials may similarly be used to create a radiopaque marker 230.

In some embodiments, the guidewire 215 can have an atraumatic straight, angled, or J-tip. The guidewire 215 can gradually transition to a stiffer segment at a proximal end. In some embodiments, the guidewire 215 can have a diameter of approximately. 0.035 in or 0.038 in. The guidewire 215 can have a variety of lengths and diameters without departing from the scope of this disclosure.

The distal end of the sheath body 222 can be configured such that when the dilator 260 is coupled to the access sheath 220 (e.g., the dilator 260 extends along the inner lumen of the access sheath 220) to form the access sheath assembly 200, the access sheath assembly 200 can be inserted smoothly over the guidewire 215 through the access site (e.g., arterial puncture) with minimal resistance. In some embodiments, the sheath body 222 can include a lubricious or hydrophilic coating to reduce friction during insertion into the vessel. For example, the coating can be limited to a distalmost section of the sheath body 222 (e.g., 0.5 centimeters (cm) to 3 cm of the elongated sheath body 222. This can facilitate insertion without compromising security of the access sheath 220 in the puncture site or the ability of the operator to firmly grasp the access sheath 220 during insertion. In some embodiments, the access sheath 220 does not include a coating. In some embodiments, the dilator 260 includes a coating, such as along distal end.

With reference to FIG. 1A, in an embodiment the access sheath 220 has features to aid in securement of the access sheath 220 during a procedure. For example the access sheath 220 may include a suture eyelet 234 or one or more ribs 236 molded into or otherwise attached to the adaptor 224 (located at the proximal end of the sheath body 222) which can allow an operator to suture tie the adaptor 224 to the patient. In some embodiments, a slidable fixture can be coupled to a part of the patient (e.g., skin) for assisting with maintaining alignment and position of the sheath body 222 relative to at least the vessel.

When the access sheath 220 is being introduced into vasculature, it is desirable to be structurally strong (e.g., resist kinking or buckling) without injuring the vasculature. For example, some procedures have limited amount of sheath insertion into the artery and/or there can be a steep angle of insertion. For example, in some instances the distal end of the sheath body 222 can be directed towards the back wall of the vessel at least during insertion into the vessel. This can cause a risk of injury from the distal end of the sheath body 222 and/or from devices being inserted through the sheath body 222. Various embodiments of access sheaths 220 and dilators 260 are described herein that are configured to provide safe, efficient, and effective access to vessels. For example, various access sheaths 220 and various dilators 260 described herein include one or more of a shorter length and an atraumatic or protective distal end for assisting with preventing unwanted damage to the vasculature, including minimizing dissection during percutaneous vessel access.

Various embodiments of an access sheath system are described below that can more safely and efficiently provide access to vasculature, such as for inserting and guiding one or more devices along the vasculature for performing various procedures.

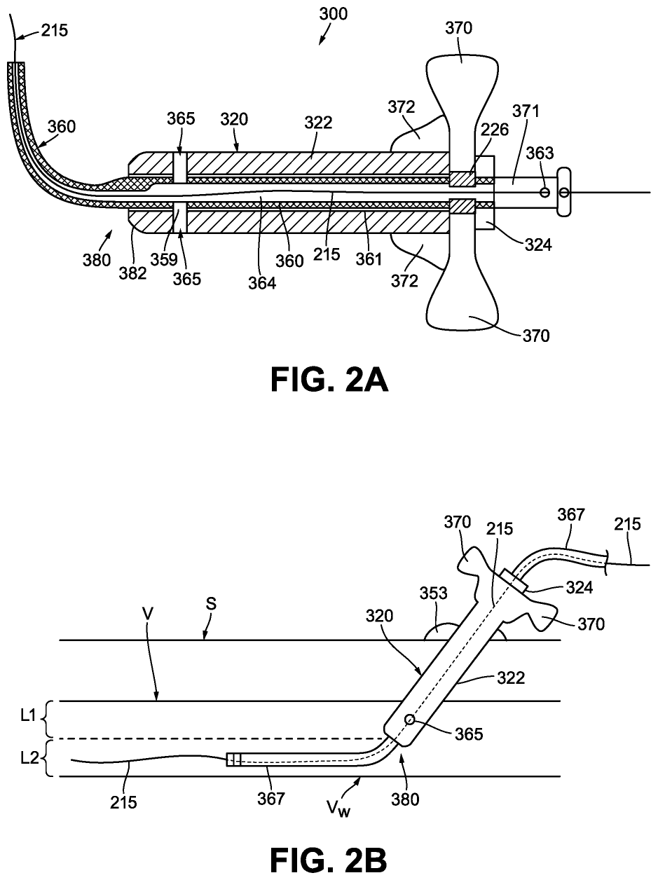

FIGS. 2A and 2B illustrate an embodiment of an access sheath system 300 for safe and effective vessel navigation and vessel puncture enlargement. As shown in FIGS. 2A and 2B, the access sheath system 300 can include an embodiment of the access sheath 320 that includes a sheath body 322 having a protective distal end 380. The access sheath 320 can be configured to protect against unwanted vessel damage. For example, in some embodiments, the access sheath body 322 can include a length that limits positioning of the protective distal end 380 within a vessel V.

As shown in FIG. 2B, in some embodiments a sheath guide 353 can be positioned along or adjacent skin S of a patient to maintain and/or control a position of the sheath body 322 relative to a vessel V. Furthermore, the sheath body 322 can include a length that causes the protective distal end 380 to extend into the vessel V without contacting or damaging an opposing vessel wall V_{w}. For example, as shown in FIG. 2B, the sheath body 322 can have a length that allows the protective distal end 380 to extend into the vessel V a first length L₁, thereby leaving approximately a second length L₂ between the protective distal end 380 of the sheath body 322 and the opposing vessel wall V_{w}. The second distance L₂ can provide space between the protective distal end 380 of the sheath body 322 and the opposing vessel wall V_{w} and prevent damage to the opposing vessel wall V_{w}. In some embodiments, the sheath body 322 can include a length between approximately 4 centimeters to approximately 10 centimeters.

As shown in FIGS. 2A and 2B, the protective distal end 380 of the sheath body 322 can include an atraumatic surface 382 that can prevent the access sheath 320 from causing harmful interaction with the vessel V, such as puncturing or damaging the opposing vessel wall V_{w}. For example the atraumatic surface 382 can be rounded and/or chamfered such that the atraumatic surface 382 does not include a sharp edge that can easily cause damage to the vessel in the event accidental or unwanted contact occurs between the opposing vessel wall V_{w} and the protective distal end 380. In some embodiments, the protective distal end 380 is made out of a flexible and/or compressible material that can reduce or prevent damage to the vessel if unintended contact is made with the protective distal end 380. For example, the protective distal end 380 can be made out of one or more of a plastic and a biocompatible material having flexible and/or compressible properties.

In some embodiments, the access sheath 320 can be guided along an embodiment of the guidewire 215 in order to position the sheath body 322, including the protective distal end 380, in a desired location. The access sheath 220 can include indicators along an outer surface, such as along an outer surface of the sheath body 322 for providing visual indication of a depth of the sheath body 322 inserted into tissue. In some embodiments, appearance of blood from an orifice of the access sheath 320 can provide visual indication that the protective distal end 380 has been inserted in the vessel V. As shown in FIG. 2A, the sheath body 322 can include a sheath inlet 365 that allows blood from the punctured vessel V to flow through the sheath inlet 365 and along an inner lumen 321 of the sheath body 322 towards a proximal end of the sheath body 322 where a user can view the blood released from the vessel. For example, once the user views blood released from the vessel, the user can identify one or more indicators along an outer surface of the sheath body 322 for positioning the protective distal end 380 of the sheath body 322 relative to the vessel. In some embodiments, the sheath inlet 365 is positioned approximately 1 millimeter to approximately 3.5 millimeters from the protective distal end 380 of the sheath body 322. By positioning the sheath inlet 365 adjacent the protective distal end 380, more accurate positioning of the protective distal end 380 can be achieved, such as relative to the vessel wall V_{w}.

As shown in FIG. 2A, an embodiment of the dilator 360 can extend through the inner lumen 361 of the access sheath 320. In some embodiments, the dilator 360 can include a dilator inlet 359, a dilator outlet 363, and an inner passageway 364 that extends between the dilator inlet 359 and dilator outlet 363. For example, when the dilator 360 is positioned in the inner lumen 361 of the access sheath 220, the dilator inlet 359 can align with the sheath inlet 365 to allow blood to flow from the vessel, through the aligned dilator inlet 359 and sheath inlet 365, and into the inner passageway 364 of the dilator 360. The blood can then flow along the inner passageway 364 of the dilator 360 and out through the dilator outlet 363, which can be positioned along a dilator cap 371, as shown in FIG. 2A. For example, during use the dilator cap 371 can be positioned outside the vessel and in view of the user such that blood reaching the dilator outlet 363 can be viewed by the user.

As shown in FIG. 2A, the sheath body 322 can include at least one arm 370 that extends out from the sheath body 322, such as to allow a user to grasp one or more arms 370. The arms 370 can be coupled to or form an extension of the sheath body 322. In one embodiment, the sheath body 322 can be configured to disassemble or separate such that the sheath body 322 can be removed with little to no disruption to devices extending through the sheath body 322. For example, a user may grasp and pull the arms 370 in a direction (e.g., away from the dilator 360 and/or longitudinal axis of the sheath body 322) that causes the sheath body 322 to retract and/or peel away (e.g., sheath body 322 separates into more than one part) from the dilator 360 and/or device extending through the sheath body 322. For example, the sheath body 322 can be formed to separate into more than one part as a result of at least one arm 370 being pulled at an angle relative to the longitudinal axis of the sheath body 322. In some embodiments, the sheath body 322 can be retracted and maintained intact, such as by pulling on the arms 370 in a proximal direction (e.g., a direction that is approximately parallel to the longitudinal axis of the sheath body 322). In some embodiments, the sheath body 322 can be formed of one or more of a polyethylene material, a polytetrafluoroethylene (PTFE) material, a fluorinated ethylene propylene (FEP) material, and a cross-linked FEP material. In some embodiments, a part of the sheath body 322 (e.g., an outer surface of the sheath body 322) can be scored and/or the material of the sheath body 322 can be cross-linked in order to allow the sheath body 322 to disassemble or separate in a particular manner and/or formation into more than one parts.

As shown in FIG. 2B, a guide sheath 367 can extend through the access sheath 320, such as to provide a protected channel between the access sheath 320 and a treatment site. The guide sheath 367 can include an elongated body with a sheath passageway extending along the elongated body. As will be described below, some embodiments of the access sheath system 300 can include an embodiment of the dilator 360 that can include an atraumatic distal tip and an embodiment of the guide sheath 367, which can combine and reduce devices of the access sheath system 300.

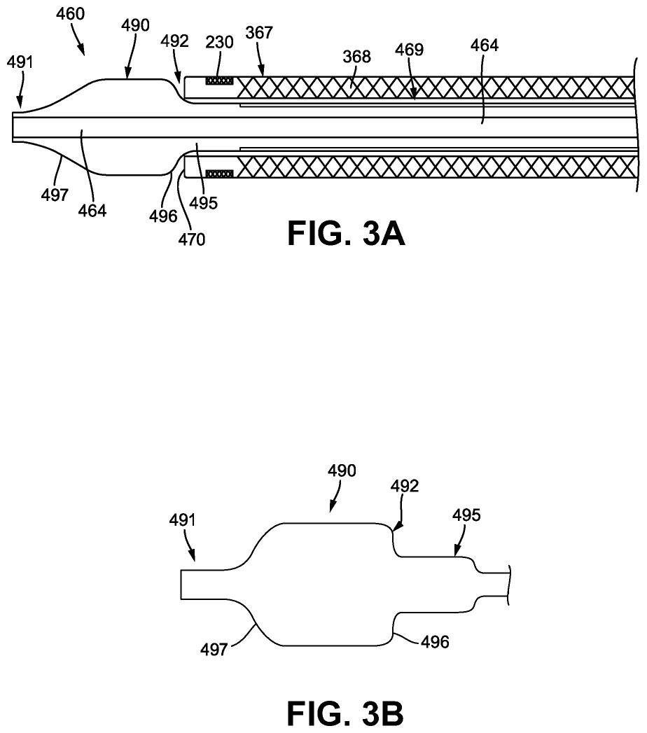

FIGS. 3A and 3B illustrate an embodiment of a dilator 460 having two parts and including an atraumatic distal tip. As shown in FIG. 3A, the dilator 460 can include an embodiment of the guide sheath 367 including an elongated body 368 and a sheath passageway 469 extending along the guide sheath 367. The dilator 460 can also have an atraumatic tip including a balloon tip element 490, as shown in FIG. 3A and 3B. For example, the balloon tip element 490 can include an inflatable tapered balloon coupled to a fluid line that allows the balloon tip element 490 to be inflated into an inflated state and deflated into a deflated state, as needed. In the inflated state, as shown in FIGS. 3A and 3B, the balloon tip element 490 can include a tapered distal surface 497 adjacent a distal end 491 of the balloon tip element 490. The tapered distal surface 497 can provide an atraumatic surface and promote safe and efficient advancement of the dilator 460 along a vessel. Additionally, the balloon tip element 490 can include a proximal engagement feature 492 that is configured to provide a protective cover over a distal end 470 of the guide sheath 367, such as to prevent vessel damage due to the guide sheath 367 being more rigid (e.g., to enable efficient vessel travel and positioning).

As shown in FIG. 3A, the proximal engagement feature 492 of the balloon tip element 490 can include a stepped proximal surface 496 that is oriented at an angle, such as approximately perpendicular, to a longitudinal axis if the dilator 460. The stepped proximal surface 496 can mate with and either fully or at least partially cover the distal end 470 of guide sheath 367 thus providing a protective cover over the distal end 470 of the guide sheath 367. The balloon tip element 490 can be made out of a variety of materials, including compliant, flexible, and/or elastic materials, which can allow the balloon tip element 490 to prevent damage to tissue during insertion into and travel along vasculature by the dilator 460. In some embodiments, the balloon tip element 490 can be inflated during insertion and/or travel of the dilator 460 and then deflated thereafter. In some embodiments, the balloon tip element 490 can be deflated and removed thereby leaving the guide sheath 367 without the balloon tip element 490 positioned within a vessel.

As shown in FIG. 3A, in some embodiments the balloon tip element 490 can include a proximal section 495 (e.g., elongated lumen) that is elongated and includes a first diameter along an outer surface of the proximal section 495 that is smaller than a second diameter of an adjacent distal section of the balloon tip element 490. The first diameter associated with the proximal section 495 can be sized to allow the proximal section 495 to extend along and have a sliding or friction fit with a sheath passageway or inner passageway 469 of the guide sheath 367. As shown in FIG. 3A, the proximal section 495 can have a length that allows the proximal section 495 to extend along the sheath passageway 469 of the guide sheath 367. This can assist with positioning and maintaining the balloon tip element 490 at the distal end of the guide sheath 367 for ensuring protection of the distal end 470 of the guide sheath 367 and preventing vessel damage.

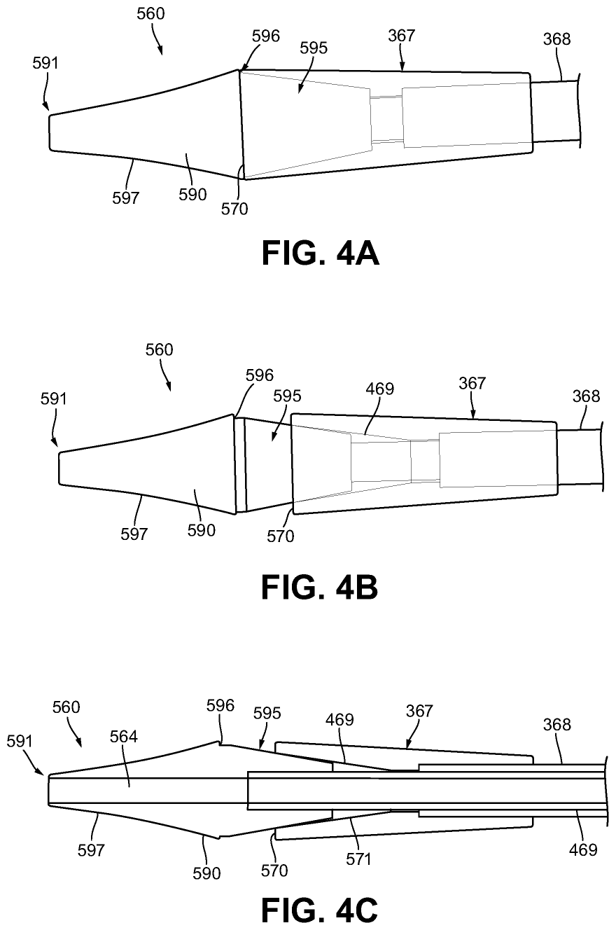

All or a part of the balloon tip element 490 can be inflatable. For example, the proximal section 495 may or may not be inflatable. In some embodiments, only a portion of the balloon tip element 490 that is distal to the proximal section 495 can be inflatable and the remaining part of the balloon tip element 490 (e.g., proximal section 495) can include a more rigid structure, such as an elongate body including a flow pathway (e.g., for inflating/deflating) and a dilator passageway or inner passageway 464 that is configured to allow a guidewire 215 to extend therealong, including along an entire length of the balloon tip 490. In some embodiments, the balloon tip element 490 can include an outer diameter (e.g., adjacent the proximal engagement feature 492) of approximately 0.103 inch to approximately 0.108 inch, such as for a 6 French guide sheath 367. In some embodiments, such as for an 8 French guide sheath 367, the outer diameter of the balloon tip element 490 can be approximately 0.124 inch to approximately 0.128 inch. For example, a length of a distal portion of the balloon tip element 490 (e.g., distal to the proximal section 495) can vary, such as between approximately 2 centimeters to approximately 12 centimeters. Other dimensions of the distal portion of the balloon tip element 490 are within the scope of this disclosure. FIGS. 4A-4C illustrate another embodiment of a dilator 560 for providing safe and effective insertion into and travel along vasculature while minimizing the number of parts and switching out of instruments in the access sheath 320. As shown in FIG. 4A, the dilator 560 can include a tapered tip element 590 that is movable along a longitudinal axis of the dilator 560 and relative to an embodiment of the guide sheath 367, which can be a part of or separate from the dilator 560. As shown in FIG. 4A, the tapered tip element 590 can include a distal tapered surface 597 adjacent a distal end 591 of the tapered tip element 590. As shown in FIG. 4B, the tapered tip element 590 can include a stepped surface 596 that is approximately perpendicular to the longitudinal axis of the dilator 560 and is configured to mate against and protect a distal end 570 of the guide sheath 367. The tapered tip element 590 can include a proximal tapered region 595 that can slidably mate with a part of the guide sheath 367, such as along a sheath passageway or inner passageway 469 of the guide sheath 367. In some embodiments, a part of the sheath passageway 469 can include a tapered or angled surface 571 that can slidably engage the proximal tapered region 595 of the tapered tip element 590, such as when the dilator 560 transitions between a first position and a second position, as shown in FIG. 4A and FIG. 4B, respectively.

For example, in the first position, as shown in FIG. 4A, the stepped surface 596 of the tapered tip element 590 can mate against and protect the distal end 570 of the guide sheath 367 from causing vessel damage, such as during insertion into and travel along a vessel. In the second position, as shown in FIGS. 4B and 4C, the stepped surface 596 of the tapered tip element 590 is positioned a distance away from the distal end 570 of the guide sheath 367. For example, the dilator 560 can form the second position when the dilator 560 is being inserted into the guide sheath 367, when the dilator 560 is being removed from the guide sheath 367, and/or when tracking the guide sheath 367 over the proximal portion of the dilator 560. As shown in FIG. 4C, the guide sheath 367 can include an elongated body 368 having a sheath passageway 469 that allows a proximal part of the tapered tip element 590 to slidably mate and extend along, such as an elongated tubular element that extends proximally and adjacent the proximal tapered region 595. The tapered tip element 590 can include a dilator passageway 564 that can allow a guidewire 215 to extend along and through the dilator 560, such as along the elongated tubular element.

For example, during use a proximal portion of the tapered tip element 590 (e.g., a proximal end of the elongated portion of the tapered tip element 590 that extends along the sheath passageway 469 of the guide sheath 367) can be advanced in a distal direction relative to the guide sheath 367 in order to advance the stepped surface 596 of the tapered tip element 590 away from the guide sheath 367, such as from the first position (FIG. 4A) to the second position (FIG. 4B). In some embodiments, the proximal portion of the tapered tip element 590 can be manipulated by a user, such as to advance the tapered tip element 590. Additionally, during use the proximal portion of the tapered tip element 590 can be advanced in a proximal direction relative to the guide sheath 367 in order to advance the stepped surface 596 of the tapered tip element 590 towards and/or against the guide sheath 367 (e.g., to at least partially cover the distal end of the guide sheath 367), such as from the second position (FIG. 4B) to the first position (FIG. 4A).

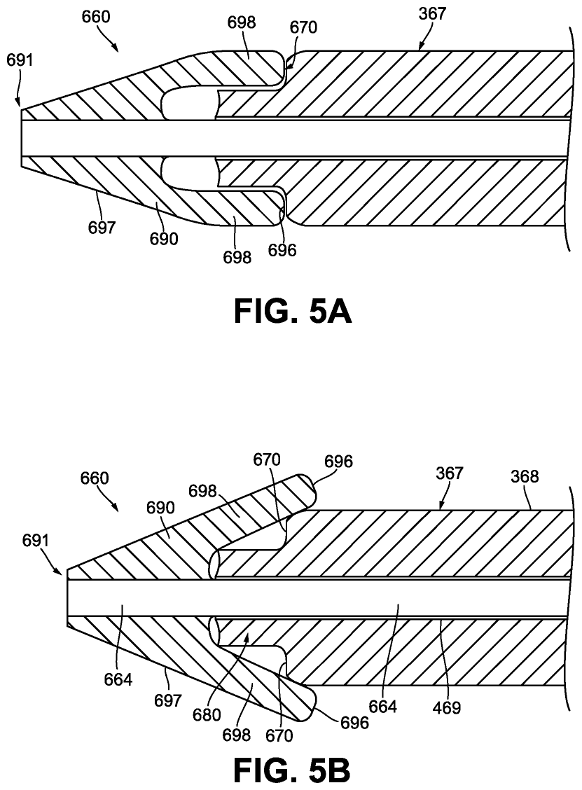

FIGS. 5A and 5B illustrate another embodiment of a dilator 660 for providing safe and effective insertion into and travel along vasculature. As shown in FIG. 5A, the dilator 660 can include a flexible tapered tip element 690 that is movable along a longitudinal axis of the dilator 660 relative to an embodiment of a guide sheath 367, which can be a part of or separate from the dilator 660. As shown in FIG. 5A, the flexible tapered tip element 690 can include a distal tapered surface 697 adjacent a distal end 691 of the flexible tapered tip element 690. As shown in FIGS. 5A and 5B, the flexible tapered tip element 690 can include flexible extensions 698 including a distal surface 696. The distal surface 696 can be approximately perpendicular to the longitudinal axis of the dilator 560 and configured to mate against and protect a distal end 570 of the guide sheath 367, such as when the dilator 660 is in a first position, as shown in FIG. 5A. The flexible tapered tip element 690 can slidably mate with a distal part of the guide sheath 367, such as when the dilator 660 transitions between a first position and a second position, as shown in FIG. 5A and FIG. 5B, respectively.

For example, in the first position (FIG. 5A), the stepped surface 696 of the flexible tapered tip element 690 can mate against and protect the distal end 670 of the guide sheath 367 from causing vessel damage, such as during insertion into and travel along a vessel. In the second position, as shown in FIG. 5B, the flexible extensions 698 can expand radially and retract proximally over the distal end 670 of the guide sheath 367. For example, the dilator 660 can form the second position when tracking the guide sheath 367 over the proximal portion of the dilator 660. As shown in FIG. 5A, some embodiments of the guide sheath 367 can include a distal extension 680 that can provide support along an inner wall of the flexible extensions 698 when the dilator 660 is in the first position, as well as limit retraction of the flexible tapered tip element 690 relative to the guide sheath 367. As shown in FIG. 5B, the guide sheath 367 can include an elongated body 368 having a sheath passageway 469 that allows a proximal part of the tapered tip element 690 to slidably mate and extend along, such as an elongated tubular element that extends proximally and adjacent the proximal tapered region 595. The tapered tip element 690 can include a dilator passageway 664 that can allow a guidewire 215 to extend along and through the dilator 660, such as along the elongated tubular element.

For example, during use a proximal portion of the tapered tip element 690 (e.g., a proximal end of the elongated portion of the tapered tip element 690 that extends along the sheath passageway 469 of the guide sheath 367) can be advanced in a distal direction relative to the guide sheath 367. Such advancement in the distal direction, such as from the second position (FIG. 5B) to the first position (FIG. 5A), can advance the stepped surface 696 of the tapered tip element 690 so that it is positioned approximately parallel relative the distal end 670 of the guide sheath 367. In some embodiments, the proximal portion (e.g., elongated lumen) of the tapered tip element 690 can be manipulated by a user, such as to advance the tapered tip element 690. Additionally, during use the proximal portion of the tapered tip element 690 can be advanced in a proximal direction relative to the guide sheath 367 in order to advance the flexible extensions 698 of the tapered tip element 690 towards and/or radially outward from the guide sheath 367, such as from the first position (FIG. 5A) to the second position (FIG. 5B). For example, the flexible extensions 698 can mate against and/or extend along an outer surface of the guide sheath 367 when the dilator is in the second position, as shown in FIG. 5B. In such a configuration, the flexible extensions can fully or at least partially cover the distal end of the guide sheath 367.

Any or all of the devices described above may be provided in kit form to the user such that one or more of the components of the systems are included in a common package or collection of packages. An embodiment of an access sheath kit comprises one or more of an access sheath, a dilator, and a guidewire that are all configured for vessel access as described above.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. An access sheath system, comprising:
an access sheath comprising an elongated sheath body that is sized and shaped to be introduced into an artery, the access sheath comprising a protective distal end having an atraumatic surface, the elongated sheath body comprising an inner lumen extending between a proximal sheath end and a distal sheath end; and
a dilator comprising an atraumatic tip and a guide sheath, the atraumatic tip having a proximal portion and a distal portion, the proximal portion of the atraumatic tip being formed to extend along a sheath passageway of the guide sheath, the distal portion of the atraumatic tip comprising a tapered distal surface, the atraumatic tip being formed to mate with and cover at least a part of a distal end of the guide sheath to protect the artery from the distal end of the guide sheath.

2. The access sheath system of claim 1, further comprising:
a guidewire sized to extend along at least a dilator passageway of the atraumatic tip.

3. The access sheath system of claim 1, wherein the atraumatic tip of the dilator includes an inflatable balloon tip element.

4. The access sheath system of claim 3, wherein the inflatable balloon tip element includes a stepped proximal surface that is formed to mate against and cover the distal end of the guide sheath when the inflatable balloon is in an inflated state.

5. The access sheath system of claim 3, wherein the inflatable balloon tip element includes a proximal section having a first outer diameter and a distal section having a second outer diameter, the first outer diameter being smaller than the second outer diameter, and the proximal section being formed to couple to the sheath passageway.

6. The access sheath system of claim 1, wherein the atraumatic tip is moveable relative to the guide sheath and along a longitudinal axis of the guide sheath for forming a first position and a second position of the dilator.

7. The access sheath system of claim 6, wherein the first position comprises a proximal surface of the atraumatic tip being mated against and at least partially covering the distal end of the guide sheath.

8. The access sheath system of claim 7, wherein the proximal surface is part of a stepped proximal surface of the atraumatic tip, the stepped proximal surface being proximal to the tapered surface.

9. The access sheath system of claim 8, wherein the stepped proximal surface is positioned a distance away from the distal end of the guide sheath when the dilator is in the second position.

10. The access sheath system of claim 6, wherein the second position comprises a flexible extension of the atraumatic tip being radially expanded and positioned along an outer surface of the distal end of the guide sheath.

11. The access sheath system of claim 1, wherein the sheath body of the access sheath includes at least one arm that, when pulled in a direction away from a longitudinal axis of the sheath body, separates the sheath body into more than one part.

12. A dilator for use with an access sheath system, the dilator comprising:
a guide sheath having a sheath passageway; and
an atraumatic tip having a proximal portion and a distal portion, the proximal portion being formed to extend along the sheath passageway, the distal portion of the atraumatic tip comprising a tapered distal surface, the atraumatic tip being formed to mate with a distal end of the guide sheath to protect an artery from the distal end of the guide sheath.

13. The dilator of claim 12, wherein the atraumatic tip includes a dilator passageway that allows a guidewire to extend therealong and through the atraumatic tip.

14. The dilator of claim 12, wherein the atraumatic tip of the dilator includes an inflatable balloon tip element.

15. The dilator of claim 14, wherein the inflatable balloon tip element includes a stepped proximal surface that is formed to mate against the distal end of the guide sheath when the inflatable balloon is an inflated state.

16. The dilator of claim 14, wherein the inflatable balloon tip element includes a proximal section having a first outer diameter and a distal section having a second outer diameter, the first outer diameter being smaller than the second outer diameter, and the proximal section being formed to couple to the sheath passageway.

17. The dilator of claim 12, wherein the atraumatic tip is moveable relative to the guide sheath and along a longitudinal axis of the guide sheath for forming a first position and a second position.

18. The dilator of claim 17, wherein the first position comprises a proximal surface of the atraumatic tip being mated against and at least partially covering the distal end of the guide sheath.

19. The dilator of claim 18, wherein the proximal surface is part of a stepped proximal surface of the atraumatic tip, the stepped proximal surface being proximal to the tapered surface.

20. The dilator of claim 19, wherein the stepped proximal surface is positioned a distance away from the distal end of the guide sheath when the dilator is in the second position.

21. The dilator of claim 17, wherein the second position comprises a flexible extension of the atraumatic tip being radially expanded and positioned along an outer surface of the distal end of the guide sheath.

22. A method, comprising:
advancing an access sheath into an artery, the access sheath comprising a protective distal end having an atraumatic surface, the elongated sheath body comprising an inner lumen extending between a proximal sheath end and a distal sheath end; and
advancing a dilator along the inner lumen of the access sheath, the dilator comprising an atraumatic tip and a guide sheath, the atraumatic tip having a proximal portion and a distal portion, the proximal portion being formed to extend along a sheath passageway of the guide sheath, the distal portion of the atraumatic tip comprising a tapered distal surface, the atraumatic tip being formed to mate with and cover at least a part of a distal end of the guide sheath to protect the artery from the distal end of the guide sheath.

23. The method of claim 22, further comprising:
advancing a guidewire along a dilator passageway of the atraumatic tip.

24. The method of claim of claim 22, wherein the atraumatic tip of the dilator includes an inflatable balloon tip element.

25. The method of claim 24, wherein the inflatable balloon tip element includes a stepped proximal surface that is formed to mate against and cover the distal end of the guide sheath when the inflatable balloon is an inflated state.

26. The method of claim 25, wherein the inflatable balloon tip element includes a proximal section having a first outer diameter and a distal section having a second outer diameter, the first outer diameter being smaller than the second outer diameter, and the proximal section coupling to the sheath passageway of the guide sheath.

27. The method of claim 22, further comprising:
moving the atraumatic tip relative to the guide sheath and along a longitudinal axis of the guide sheath for forming a first position or a second position.

28. The method of claim 27, wherein the first position comprises a proximal surface of the atraumatic tip being mated against and at least partially covering the distal end of the guide sheath.

29. The method of claim 28, wherein the proximal surface is part of a stepped proximal surface of the atraumatic tip, the stepped proximal surface being proximal to the tapered surface.

30. The method of claim 29, wherein the stepped proximal surface is positioned a distance away from the distal end of the guide sheath when the dilator is in the second position.

31. The method of claim 27, wherein the second position comprises a flexible extension of the atraumatic tip being radially expanded and positioned along an outer surface of the distal end of the guide sheath.

32. The method of claim 22, wherein the sheath body of the access sheath includes at least one arm that, when pulled in a direction away from a longitudinal axis of the sheath body, separates the sheath body into more than one part.
